(19) United States
(12) Patent Application Publication
(10) Pub. No.: US 2013/0123144 A1
(43) Pub. Date: May 16, 2013

(54) TUNABLE LCST POLYMERS AND METHODS OF PREPARATION

(71) Applicants: David Putnam, Ithaca, NY (US); Lihong Huang, Cary, NC (US)

(72) Inventors: David Putnam, Ithaca, NY (US); Lihong Huang, Cary, NC (US)

(73) Assignee: CORNELL UNIVERSITY, Ithaca, NY (US)

(21) Appl. No.: 13/696,365

(22) PCT Filed: May 6, 2011
(86) PCT No.: PCT/US11/35453

§ 371 Date: Jan. 29, 2013

Related U.S. Application Data
(60) Provisional application No. 61/331,987, filed on May 6, 2010.

(51) Int. Cl. C08G 61/04; G01N 21/27; C07C 233/47

(52) U.S. Cl. CPC C08G 61/04; C07C 233/47; G01N 21/27
USPC 506/12; 562/567; 528/331; 525/421; 506/20

(57) ABSTRACT

Polymer compositions having the chemical structure: as well as monomer compositions for producing said polymers are described. Methods for preparing these polymers and combinatorial libraries of these polymers are also described.

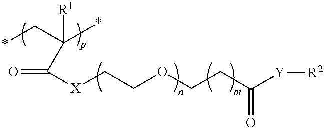

### CROSS REFERENCE TO RELATED APPLICATION

This application claims the benefit of priority from U.S. Provisional Application No. 61/331,987, filed on May 6, 2010.

### GOVERNMENT SUPPORT

This invention was made with government support under CBET-0642509 awarded by the National Science Foundation and a Grant from the Morgan Tissue Engineering Fund. The government has certain rights in the invention.

### FIELD OF THE INVENTION

The present invention relates generally to lower critical solution temperature (LCST) polymer compositions, as well as methods for their synthesis and use.

### BACKGROUND OF THE INVENTION

LCST polymers exhibit a sudden volume phase transition at a critical (i.e., LCST) temperature in aqueous solution. When the temperature is raised above the LCST, the polymer chain assumes a sudden increase in hydrophobicity, which makes the polymer substantially insoluble in aqueous solution. This unique property of LCST polymers makes them of particular interest in such applications as agents for controlling or modifying bacterial aggregation, protein adsorption and release, protein-ligand recognition, and drug delivery.

However, a significant obstacle being encountered in their integration into these and future applications is their substantial incapacity in being derivatized by any of a diverse selection of functional groups. Without this ability, the utility of this class of polymers, as well as the conditions in which they can be used, are highly limited. For example, the properties of existing LCST polymers are generally very difficult to precisely tune since fine adjustments to their structure is generally not possible. Some particular LCST polymers that lack this ability are the poly(N-substituted)acrylamides. Hence, there would be a significant advantage in LCST polymers that are tunable in such properties as critical temperature and interaction ability with a host, by appropriate fine adjustment in their structure.

### SUMMARY OF THE INVENTION

The invention is directed, in a first aspect, to monomer compositions useful in the preparation of LCST polymers described herein. In particular embodiments, the monomer compositions are represented by the following chemical structure:

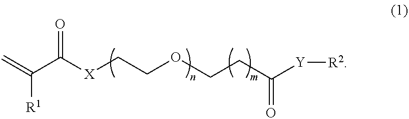

In Formula (1), R¹ and R² are independently selected from a hydrogen atom or a hydrocarbon group containing at least one carbon atom; X represents an —O— or —NR⁵—group; and Y represents an —O—, —S—, or —NR³R⁴— group. The substituents R³, R⁴, and R⁵ independently represent a hydrogen atom or a hydrocarbon group containing at least one carbon atom, except that one of R³ and R⁴ can instead represent an unshared pair of electrons. The subscript n represents an integer of at least 1, 2, 3, or 4, and the subscript m represents 0 or an integer of at least 1, 2, or 3.

In a second aspect, the invention is directed to a polymer composition derived by polymerization of any of the monomer compositions described above. In particular embodiments, the polymer composition is represented by the following chemical structure:

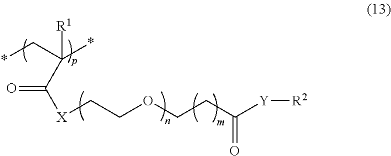

In Formula (13), R¹, R², X, Y, R³, R⁴, R⁵, m, and n are as defined above for the monomer composition. The subscript p represents an integer of at least 2.

In a third aspect, the invention is directed to a method for producing a polymer according to the above polymeric formula. The method involves polymerizing a monomer composition described above by any suitable method. In particular embodiments, the polymerization method is a RAFT or ATRP polymerization method.

In a fourth aspect, the invention is directed to a combinatorial library of LCST polymers in which the polymers in the library vary in one or more variables selected from X, Y, R¹, R², n, m, and p. Related to this embodiment is a method for high-throughput screening of the combinatorial library of LCST polymers in order to efficiently elucidate their LCST and other properties.

### BRIEF DESCRIPTION OF THE FIGURES

FIGS. 1A,B. Schemes showing (A) synthesis of monomer CTMAAm (iii) and polymerization by RAFT, and (B) structure of brush-type polymer pCTMAAm (vi).

FIG. 2A,B. Graphs showing (A) relationship of Mₙ experimental (GPC), Mₙ theoretical, and PDI (GPC) with conversion of CTMAAm RAFT polymerization at [M]₀/[CTA]₀/[I]₀=200:1:0.25, and (B) pseudo-first-order kinetic plot with various ratios of [M]₀/[CTA]₀/[I]₀. Dashed lines represent linear regressions calculated based on short times only where chain growth is linear with time.

FIG. 3. Scheme showing preparation of a polymer library, wherein systematic variation in structural parameters is achieved by using pCTMAAm (with hydrophilic carboxylic acid endcapping groups) as template and replacing a portion of carboxylic acid endcapping groups therein with hydrophobic N-substituted amide groups (—NHR, where R is an alkyl group).

FIG. 4. Graph showing temperature dependence of transmittance at 500 nm for 3 mg/mL of solutions of polymers in the polymer library varying in propyl, butyl, and hexyl endcapping groups.

FIGS. 5A-C. Graphs showing the substitution dependence of LCST polymers varying in propyl, butyl, and hexyl endcapping groups.

FIGS. 6A-C. Graphs showing the pH dependence of LCST polymers varying in propyl, butyl, and hexyl endcapping groups.

FIG. 7. Three-phase diagram showing dependence of LCST with three parameter spaces, including substitution, molecular weight of polymer, and carbon number of conjugation group, in a library of LCST polymers.

### DETAILED DESCRIPTION OF THE INVENTION

For convenience, before further description of the present invention, certain terms employed in the specification, examples, and appended claims are described here. These definitions should be read in light of the entire disclosure and as would be understood by a person skilled in the art.

The terms “hydrocarbon group” and “hydrocarbon linker”, as used herein, are, in a first embodiment, composed solely of carbon and hydrogen. In different embodiments, one or more of the hydrocarbon groups or linkers can contain precisely, or a minimum of, or a maximum of, for example, one, two, three, four, five, six, seven, eight, nine, ten, eleven, twelve, thirteen, fourteen, fifteen, sixteen, seventeen, eighteen, nineteen, or twenty carbon atoms, or a number of carbon atoms within a particular range bounded by any two of the foregoing carbon numbers. Hydrocarbon groups or linkers in different compounds described herein, or in different positions of a compound, may possess the same or different number (or preferred range thereof) of carbon atoms in order to independently adjust or optimize the activity or other characteristics of the compound.

The hydrocarbon groups or linkers can be, for example, saturated and straight-chained (i.e., straight-chained alkyl groups or alkylene linkers). Some examples of straight-chained alkyl groups (or alkylene linkers) include methyl (or methylene linker, i.e., —CH₂—, or methine linker), ethyl (or ethylene or dimethylene linker, i.e., —CH₂CH₂— linker), n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, and n-eicosyl groups (or their respective linker analogs).

The hydrocarbon groups or linkers can alternatively be saturated and branched (i.e., branched alkyl groups or alkylene linkers). Some examples of branched alkyl groups include isopropyl, isobutyl, sec-butyl, t-butyl, isopentyl, neopentyl, 2-methylpentyl, 3-methylpentyl, and the numerous C₇, C₈, C₉, C₁₀, C₁₁, C₁₂, C₁₃, C₁₄, C₁₅, C₁₆, C₁₇, C₁₈, C₁₉, and C₂₀ saturated and branched hydrocarbon groups. Some examples of branched alkylene linkers are those derived by removal of a hydrogen atom from one of the foregoing exemplary branched alkyl groups (e.g., isopropylene, —CH(CH₃)CH₂—).

The hydrocarbon groups or linkers can alternatively be saturated and cyclic (i.e., cycloalkyl groups or cycloalkylene linkers). Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane). Some examples of cycloalkylene linkers are those derived by removal of a hydrogen atom from one of the foregoing exemplary cycloalkyl groups.

The hydrocarbon groups or linkers can alternatively be unsaturated and straight-chained (i.e., straight-chained olefinic or alkenyl groups or linkers). The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), and the numerous C₇, C₈, C₉, C₁₀, C₁₁, C₁₂, and higher unsaturated and straight-chained hydrocarbon groups. Some examples of straight-chained olefinic linkers are those derived by removal of a hydrogen atom from one of the foregoing exemplary straight-chained olefinic groups (e.g., vinylene, —CH═CH—, or vinylidene).

The hydrocarbon groups or linkers can alternatively be unsaturated and branched (i.e., branched olefinic or alkenyl groups or linkers). Some examples of branched olefinic groups include propen-2-yl (CH₂═C.—CH₃), 3-buten-2-yl (CH₂═CH—CH.—CH₃), 3-buten-3-yl (CH₂═C.—CH₂—CH₃), 4-penten-2-yl, 4-penten-3-yl, 3-penten-2-yl, 3-penten-3-yl, 2,4-pentadien-3-yl, and the numerous C₆, C₇, C₈, C₉, C₁₀, C₁₁, C₁₂, and higher unsaturated and branched hydrocarbon groups. Some examples of branched olefinic linkers are those derived by removal of a hydrogen atom from one of the foregoing exemplary branched olefinic groups.

The hydrocarbon groups or linkers can alternatively be unsaturated and cyclic (i.e., cycloalkenyl groups or cycloalkenylene linkers). The unsaturated and cyclic group can be aromatic or aliphatic. Some examples of unsaturated and cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group can also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene. Some examples of cycloalkenylene linkers are those derived by removal of a hydrogen atom from one of the foregoing exemplary cycloalkenyl groups (e.g., phenylene and biphenylene).

One or more of the hydrocarbon groups or linkers may also include one or more heteroatoms (i.e., non-carbon and non-hydrogen atoms), such as one or more heteroatoms selected from oxygen, nitrogen, sulfur, and halide atoms, as well as groups containing one or more of these heteroatoms (i.e., heteroatom-containing groups). Some examples of oxygen-containing groups include hydroxy (OH), carbonyl-containing (e.g., carboxylic acid, ketone, aldehyde, carboxylic ester, amide, and urea functionalities), nitro (NO₂), carbon-oxygen-carbon (ether), sulfonyl, and sulfinyl (i.e., sulfoxide), and amine oxide groups. The ether group can also be a polyalkyleneoxide group, such as a polyethyleneoxide group. Some examples of nitrogen-containing groups include primary amine, secondary amine, tertiary amine, quaternary amine, cyanide (i.e., nitrile), amide (i.e., —C(O)NR₂ or —NRC(O)R, wherein R is independently selected from hydrogen atom and hydrocarbon group, as described above), nitro, urea, imino, and carbamate, wherein it is understood that a quaternary amine group necessarily possesses a positive charge and requires a counteranion. Some examples of sulfur-containing groups include mercapto (i.e., —SH), thioether (i.e., sulfide), disulfide, sulfoxide, sulfone, sulfonate, and sulfate groups. Some examples of halide atoms considered herein include fluorine, chlorine, and bromine. One or more of the heteroatoms described above (e.g., oxygen, nitrogen, and/or sulfur atoms) can be inserted between carbon atoms (e.g., as —O—, —NR—, or —S—) in any of the hydrocarbon groups described above to form a heteroatom-substituted hydrocarbon group or linker. Alternatively, or in addition, one or more of the heteroatom-containing groups can replace one or more hydrogen atoms on the hydrocarbon group or linker.

In particular embodiments, the hydrocarbon group is, or includes, a cyclic group. The cyclic hydrocarbon group may be, for example, monocyclic by containing a single ring without connection or fusion to another ring. The cyclic hydrocarbon group may alternatively be, for example, bicyclic, tricyclic, tetracyclic, or a higher polycyclic ring system by having at least two rings interconnected and/or fused.

In some embodiments, the cyclic hydrocarbon group is carbocyclic, i.e., does not contain ring heteroatoms (i.e., only ring carbon atoms). In different embodiments, ring carbon atoms in the carbocyclic group are all saturated, or a portion of the ring carbon atoms are unsaturated, or the ring carbon atoms are all unsaturated (as found in aromatic carbocyclic groups, which may be monocyclic, bicyclic, tricyclic, or higher polycyclic aromatic groups).

In some embodiments, the hydrocarbon group is, or includes, a cyclic or polycyclic group that includes at least one ring heteroatom (for example, one, two, three, four, or higher number of heteroatoms). Such ring heteroatom-substituted cyclic groups are referred to herein as “heterocyclic groups”. As used herein, a “ring heteroatom” is an atom other than carbon and hydrogen (typically, selected from nitrogen, oxygen, and sulfur) that is inserted into, or replaces a ring carbon atom in, a hydrocarbon ring structure. In some embodiments, the heterocyclic group is saturated, while in other embodiments, the heterocyclic group is unsaturated (i.e., aliphatic or aromatic heterocyclic groups, wherein the aromatic heterocyclic group is also referred to herein as a “heteroaromatic ring”, or a “heteroaromatic fused-ring system” in the case of at least two fused rings, at least one of which contains at least one ring heteroatom). In some embodiments, the heterocyclic group is bound via one of its ring carbon atoms to another group (i.e., other than hydrogen atom and adjacent ring atoms), while the one or more ring heteroatoms are not bound to another group. In other embodiments, the heterocyclic group is bound via one of its heteroatoms to another group, while ring carbon atoms may or may not be bound to another group.

Some examples of saturated heterocyclic groups include those containing at least one oxygen atom (e.g., oxetane, tetrahydrofuran, tetrahydropyran, 1,4-dioxane, 1,3-dioxane, and 1,3-dioxepane rings), those containing at least one nitrogen atom (e.g., pyrrolidine, piperidine, piperazine, imidazolidine, azepane, and decahydroquinoline rings), those containing at least one sulfur atom (e.g., tetrahydrothiophene, tetrahydrothiopyran, 1,4-dithiane, 1,3-dithiane, and 1,3-dithiolane rings), those containing at least one oxygen atom and at least one nitrogen atom (e.g., morpholine and oxazolidine rings), those containing at least one oxygen atom and at least one sulfur atom (e.g., 1,4-thioxane), and those containing at least one nitrogen atom and at least one sulfur atom (e.g., thiazolidine and thiamorpholine rings).

Some examples of unsaturated heterocyclic groups include those containing at least one oxygen atom (e.g., furan, pyran, 1,4-dioxin, and dibenzodioxin rings), those containing at least one nitrogen atom (e.g., pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, 5,6,7,8-tetrahydroquinoxaline, 1,8-naphthyridine, and 4-azabenzimidazole rings), those containing at least one sulfur atom (e.g., thiophene, thianaphthene, and benzothiophene rings), those containing at least one oxygen atom and at least one nitrogen atom (e.g., oxazole, isoxazole, benzoxazole, benzisoxazole, oxazoline, 1,2,5-oxadiazole (furazan), and 1,3,4-oxadiazole rings), and those containing at least one nitrogen atom and at least one sulfur atom (e.g., thiazole, isothiazole, benzothiazole, benzoisothiazole, thiazoline, and 1,3,4-thiadiazole rings).

In one aspect, the invention is directed to a vinylic monomer composition represented by the following chemical structure:

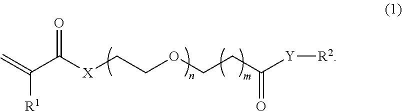

In Formula (1), R¹ and R² are independently selected from a hydrogen atom or a hydrocarbon group containing at least one carbon atom. Typically, R¹ is a hydrogen atom or methyl group. In particular embodiments, R² is a straight-chained or branched alkyl group of at least one, two, three, four, or five carbon atoms and up to six, seven, eight, nine, ten, eleven, or twelve carbon atoms. In other particular embodiments, R² is a carbocyclic group, which may be a saturated cyclic group, aliphatic cyclic group, or aromatic group. X represents an —O— or —NR⁵— group, and Y represents an —O—, —S—, or —NR³R⁴— group (wherein the dashes in —NR³R⁴— indicate linking at the N atom only), wherein R³, R⁴, and R⁵ independently represent a hydrogen atom or a hydrocarbon group containing at least one carbon atom. As hydrocarbon groups, R³, R⁴, and R⁵ are typically straight-chained or branched alkyl groups containing one, two, three, or four carbon atoms. Although both of R³ and R⁴ can be selected from hydrogen atom and hydrocarbon groups (resulting in an ammonium linker), typically, one of R³ and R⁴ is an unshared pair of electrons. The subscript n represents an integer of at least 1. In different embodiments, n is precisely, at least, up to, or less than, for example, 1, 2, 3, 4, 5, 6, 7, 8, 9, 10, 11, or 12, or a number within a range bounded by any two of the foregoing values. The subscript m represents 0 or an integer of at least 1. In different embodiments, m is precisely, at least, up to, or less than, for example, 1, 2, 3, 4, 5, 6, 7, 8, 9, 10, 11, or 12, or a number within a range bounded by any two of the foregoing values. In other embodiments, n and m sum to precisely or at least 1, 2, 3, 4, 5, 6, 7, 8, 9, 10, 11, or 12, or a sum within a range bounded by any two of the foregoing values.

When Y is an —O— or —S— group, the bond shown between R² and Y is either a covalent or ionic bond. If the bond is ionic, R² is an organic or inorganic cationic group that counterbalances a negative charge on Y, as in the case of carboxylate or thiocarboxylate salt of a metal ion (e.g., Na⁺) or ammonium ion (e.g., ammonium, trimethylammonium, or tetramethylammonium ion). In other embodiments, the group —Y—R² can, itself, be a cationic group that is necessarily associated with an anionic counterion (not denoted in Formula I), as in the case where the group —Y—R² represents a —NR³R⁴R⁵ group, where R³, R⁴, and R⁵ are selected from hydrogen atom and/or hydrocarbon group. The monomer composition shown in Formula (1) may contain other ionic portions not shown. Any one or more ionic groups in Formula (1) results in a salt of the monomer composition.

In a first set of embodiments of Formula (1), the monomer composition has a structure according to the following formula:

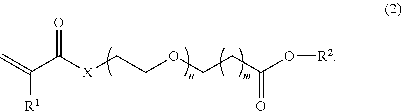

In a second set of embodiments of Formula (1), the monomer composition has a structure according to the following formula:

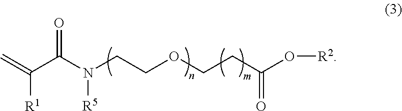

In a third set of embodiments of Formula (1), the monomer composition has a structure according to the following formula:

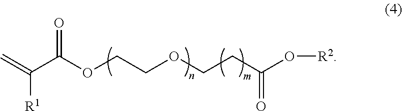

In a fourth set of embodiments of Formula (1), the monomer composition has a structure according to the following formula:

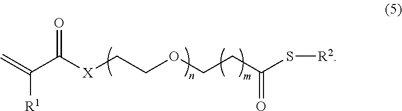

In a fifth set of embodiments of Formula (1), the monomer composition has a structure according to the following formula:

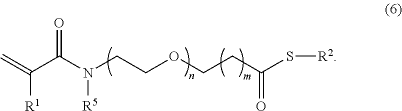

In a sixth set of embodiments of Formula (1), the monomer composition has a structure according to the following formula:

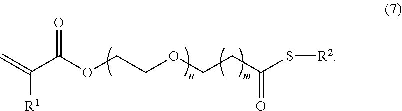

In a seventh set of embodiments of Formula (1), the monomer composition has a structure according to the following formula:

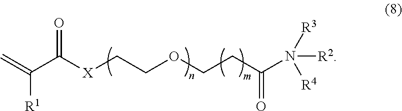

In an eighth set of embodiments of Formula (1), the monomer composition has a structure according to the following formula:

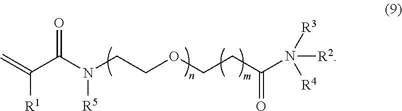

In a ninth set of embodiments of Formula (1), the monomer composition has a structure according to the following formula:

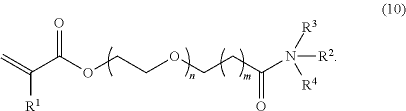

In a tenth set of embodiments of Formula (1), the monomer composition has a structure according to the following formula:

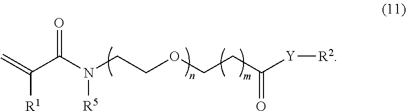

In an eleventh set of embodiments of Formula (1), the monomer composition has a structure according to the following formula:

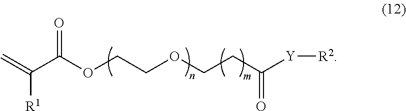

In Formulas (2)-(12), R¹, R², R³, R⁴, R⁵, X, Y, n, and m are as defined above, including any of the particular embodiments described above for these groups or variables. Moreover, for Formulas (8)-(10), if one of R², R³, or R⁴ is an unshared pair of electrons, the group —NR²R³R⁴ can be replaced with —NR²R³ in any of these formulas.

For any of Formulas (1)-(12) above, the group R², or —YR², in particular, can also be a biologically relevant species. The biologically relevant species can be, for example, a molecule or macromolecule derived from a living organism, or that mimics a biological molecule or macromolecule found in a living organism. The biologically relevant species may, for example, target or modulate a molecule, macromolecule, or process in a biological material or living organism. The target may be, for example, a cell membrane, organelle, or cytoplasmic molecule of a cell. The purpose of targeting may be, for example, to modulate a protein function, or to modulate or regulate a gene expression, or to contact the target with another chemical species (e.g., a pharmaceutical) contained anywhere in the composition shown in Formula (1). In various embodiments, R² is a biologically relevant species that is, or includes, for example, a peptide, dipeptide, tripeptide (e.g., glutathione), tetrapeptide, pentapeptide, hexapeptide, higher oligopeptide, protein, monosaccharide, disaccharide, trisaccharide, tetrasaccharide, higher oligosaccharide, polysaccharide (e.g., a carbohydrate), nucleobase, nucleoside (e.g., adenosine, cytidine, uridine, guanosine, thymidine, inosine, and S-Adenosyl methionine), nucleotide (i.e., mono-, di-, or tri-phosphate forms), dinucleotide, trinucleotide, tetranucleotide, higher oligonucleotide, nucleic acid (e.g., DNA, sRNA, tRNA, mRNA, or a plasmid), cofactor (e.g., TPP, FAD, NAD, coenzyme A, biotin, lipoamide, metal ions (e.g., Mg²⁺), metal-containing clusters (e.g., the iron-sulfur clusters), or a non-biological (i.e., synthetic) targeting group. Some particular types of proteins include enzymes, hormones, antibodies (e.g., monoclonal antibodies), lectins, and steroids. The antibody can be a whole antibody, or alternatively, a fragment of an antibody that retains the recognition portion (i.e., hypervariable region) of the antibody. Some examples of antibody fragments include Fab, Fc, and F(ab′)₂ fragments.

The conjugation of biological species to other biological species or to non-biological materials is well-known in the art. R² or —YR² can be reacted directly or via a double-reactive linker to bond with a biological material. To bind with a biological material, R² or —YR² is or includes, or is appropriately modified to possess, one or more groups reactive with one or more groups on the biological material. For example, —C(O)YR² in Formula (1) may be selected as a —COOH or —COOR² group, where R² is a group that results in an activated ester (e.g., succinimide or other activating group), and the acid or activated ester of Formula (1) is reacted, under conditions well-known in the art, with an amino-containing species (e.g., a peptide, protein, or nucleic acid) to form an amide linkage with said species. As another example, —YR² in Formula (1) may be taken as a chlorine atom so that Formula (1) is an acyl chloride, which can then be reacted with an amino-containing species. As another example, R² may be selected as an alkyl group containing an accessible reactive group (e.g., where R² is —(CH₂)ₙ—R^{2′}, where R^{2′} is a reactive group and n is as defined above), wherein the reactive group may be, for example, a hydroxy group, amino group, thiol group, bromo atom, or iodo atom. Numerous double-reactive linkers are known that can link any such reactive groups with one or more active groups on the biological material. Some double-reactive linkers include amino-amino couplers (e.g., linkers bearing two activated ester groups), amino-thiol couplers (e.g., linkers bearing an activated ester group on one end and a thiol-reactive group (e.g., maleimido) on the other end), carboxy-amino couplers, hydroxy-amino couplers, carboxy-thiol couplers, and thiol-thiol couplers.

In some embodiments of any of Formulas (I-12), any one or more of the following monomer compositions can be excluded:

(i) a monomer composition according to Formula (1) wherein X is NH, R¹ is methyl, n is 3, m is 0, Y is O, and R² is H;

(ii) a monomer composition according to Formula (1) wherein X is NH, R¹ is methyl, n is 3, m is 0, Y is O, and R² is ethyl;

(iii) a monomer composition according to Formula (1) wherein X is O, R¹ is methyl, n is 1, m is 0, Y is O, and R² is comprised of an adamantyl group;

(iv) a monomer composition according to Formula (1) wherein X is O, R¹ is H, n is 1, m is 0, Y is O, and R² is comprised of an adamantyl group;

(v) a monomer composition according to Formula (1) wherein X is O, R¹ is H, n is 3, m is 1, Y is O, and R² is t-butyl;

(vi) a monomer composition according to Formula (1) wherein X is O, R¹ is methyl, n is 1, m is 0, Y is O, and R² is methyl;

(vii) a monomer composition according to Formula (1) wherein X is O, R¹ is H, n is 1, m is 4, Y is O, and R² is comprised of a phenyl ring bound to a tetrahydropyran ring;

(viii) a monomer composition according to Formula (1) wherein X is O, R¹ is H, n is 1, m is 4, Y is O, and R² is a hydroxyphenyl group;

(ix) a monomer composition according to Formula (1) wherein X is O, R¹ is H, n is 1, m is 4, Y is O, and R² is comprised of a phenyl ring bound to a carboxylcyclohexyl group;

(x) a monomer composition according to Formula (1) wherein X is O, R¹ is H, n is 1, m is 0, Y is O, and R² is comprised of an oxetane ring;

(xi) a monomer composition according to Formula (1) wherein X is O, R¹ is H, n is 1, m is 0, Y is O, and R² is methyl;

(xii) a monomer composition according to Formula (1) wherein X is O, R¹ is H, n is 1, m is 4, Y is O, and R² is (CH₂)₅COOH;

(xiii) a monomer composition according to Formula (1) wherein X is O, R¹ is H, n is 1, m is 0, Y is O, and R² is a benzaldehyde group;

(xiv) a monomer composition according to Formula (1) wherein X is NH, R¹ is H, n is 3, m is 0, Y is O, and R² is t-butyl;

(xv) a monomer composition according to Formula (1) wherein X is O, R¹ is methyl, n is 1, m is 4, Y is O, and R² is n-octyl; and

(xvi) a monomer composition according to Formula (1) wherein X is O, R¹ is H, n is at least l, m is 0, Y is O, and R² is comprised of an N-bound succinimide group.

In some embodiments, one or more of R², R³, and R⁴ is a hydrocarbon group substituted by at least one hydrophilic group. Some examples of hydrophilic groups include amino, imino, amido, hydroxyl, ether, polyether, carboxyl, ester (which can be an inorganic ester, organoester, or thioester), carbamato, ureido, aldehydro, keto, sulfate, sulfonate, sulfone, sulfoxide, sulfite, phosphate, phosphonate, phosphinate, phosphite, nitro, nitroso, and charged groups. In other embodiments, at least one of R², R³, and R⁴ is a hydrocarbon group that contains solely carbon and hydrogen atoms, and may or may not also include one or more halogen atoms. In yet other embodiments, at least one of R², R³, and R⁴ is an amphiphilic group by possessing a hydrophobic moiety and a hydrophilic moiety. Generally, the hydrophobic portion of the amphiphilic group contains at least three, four, five, or six interlinked carbon atoms with only hydrogen atoms attached to the carbon atoms. Other variable groups (i.e., R¹ and/or R⁵) may also include a hydrophilic group, or instead be composed solely of carbon and hydrogen, which may or may not also include one or more halogen atoms, or instead be an amphiphilic group.

In another aspect, the invention is directed to polymers that include addition units of any of the monomer compositions described above. As understood in the art, by being “addition units” is meant that the vinyl-containing monomer compositions described herein polymerize, under conditions well known in the art, via repetitive linkage of vinyl carbon atoms. In one set of embodiments, the polymer is a homopolymer by being constructed of only one type of monomer structure, selected from any of the monomer structures described above. In another set of embodiments, the polymer is a copolymer, which can be, for example, a binary, ternary, or quaternary copolymer. Furthermore, the copolymer can have any known arrangement, such as block, random, alternating, and graft arrangements. In one set of embodiments, the copolymer is constructed solely of two or more of the monomer compositions described above. In other embodiments, the copolymer is constructed of at least one of the monomer compositions described above and monomer compositions not described above. Some examples of other monomer compositions that may be included in the copolymer composition include any vinyl-containing species capable of undergoing an addition reaction, such as acrylic acid, methacrylic acid, hydrocarbon ester derivatives thereof (e.g., methyl acrylate, ethyl acrylate, n-propyl acrylate, methyl methacrylate, ethyl methacrylate, n-propyl methacrylate), acrylamide and N- or N,N-hydrocarbon derivatives thereof (e.g., N-methylacrylamide, N,N-dimethylacrylamide, N-ethylacrylamide, N,N-diethylacrylamide, N-butylacrylamide), styrene, p-hydroxystyrene, p-vinylbenzoic acid, and vinyl acetate. The other monomers may also contain reactive groups useful for further structural modification or conjugation to other groups or chemical entities. Some examples of reactive groups include carboxy, carboxy ester, amino, haloalkyl, cyclic ether, and mercapto groups.

The polymers described above may be conveniently depicted by the following formula:

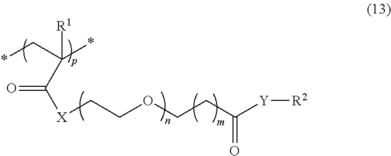

In Formula (13), X, Y, n, m, R, and R² are all as defined above. The variable p is preferably at least 10 (i.e., at least 10 monomer units). In some embodiments, p can be at least 20, 50, 100, 500, or 1000. In other embodiments, p corresponds to a weight average molecular weight (M_{w}) of the polymer, e.g., a M, of at least 1000, 5000, 10,000, 50,000, 100,000, or greater. In embodiments where the polymer of Formula (13) is a homopolymer, the polymer contains solely one type of repeating unit according to Formula (13) wherein the variables X, Y, n, m, R¹, and R² are the same from unit to unit. In one set of embodiments where the polymer of Formula (13) represents a copolymer, the copolymer is constructed solely of p monomer units depicted in Formula (13), provided that at least one of the variables X, Y, n, m, R, and R² is not the same from unit to unit. In another set of embodiments where the polymer of Formula (13) represents a copolymer, the copolymer is constructed of p monomer units depicted in Formula (13) as well as any number of monomer units not depicted in Formula (13). The copolymers can be alternatively depicted as having p1 and p2 different monomer units (for a binary copolymer), or p1, p2, and p3 different monomer units (for a ternary polymer), wherein it is understood that the sum of p1 and p2, or the sum of p1, p2, and p3, is p. The polymer may have any suitable polydispersity value, such as a value of or less than 2, 1.5, 1.4, 1.3, 1.2, or 1.1, or a value of or greater than 1, 1.2, 1.5, 1.7, or 2.

In a first set of embodiments, the polymer according to Formula (13) has a structure according to the following formula:

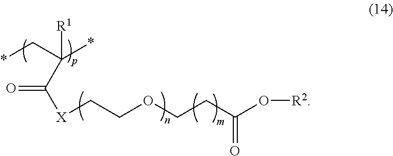

In a second set of embodiments, the polymer according to Formula (13) has a structure according to the following formula:

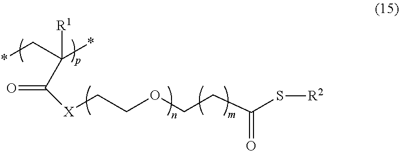

In a third set of embodiments, the polymer according to Formula (13) has a structure according to the following formula:

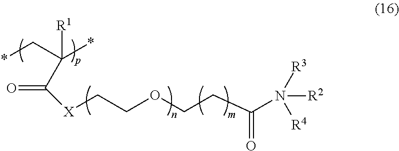

In a fourth set of embodiments, the polymer according to Formula (13) has a structure according to the following formula:

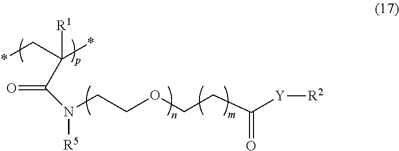

In a fifth set of embodiments, the polymer according to Formula (13) has a structure according to the following formula:

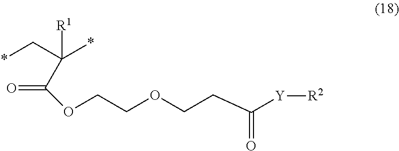

In Formulas (13)-(18), R¹, R², R³, R⁴, R⁵, X, Y, n, m, and p are as defined above, including any of the particular embodiments described above for these groups or variables. Moreover, any one or more of the exclusions provided above for the monomer compositions may also be applied to any of the polymer compositions described above.

In some embodiments where general Formula (13) represents a copolymer containing at least two different types of monomer units selected from any of the monomer compositions described above (i.e, under Formulas I-12), —YR² or R² is at least one varying structural feature from unit to unit. In these embodiments, Y may be the same or different between different types of monomer units, while R² may independently be the same or different between different types of monomer units. In one particular set of embodiments, a portion of the monomer units have R² as H while a portion of the monomer units have R² as a hydrocarbon group. For example, —YR² may represent —OH (i.e., a carboxyl endcapping group) for a portion of the monomer units, and —YR² may represent a —OR² group wherein R² is a hydrocarbon group (i.e., a carboxy ester endcapping group) for another portion of the monomer units, wherein the hydrocarbon group is, for example, a straight-chained or branched alkane having at least one, two, or three carbon atoms and up to four, five, six, seven, eight, nine, ten, eleven, or twelve carbon atoms. In the foregoing example, the same principle is applied to the situation where Y is S or —NR³R⁴, or where the different monomer units have different Y groups. Some other examples of copolymers include the situation where a portion of the monomer units have —YR² as —OR² (where R² is H or a hydrocarbon group), and another portion of the monomer units have —YR² as —SR² or —NR³R⁴R⁵. As yet another example of a copolymer, a portion of the monomer units may have —YR² as —SR² (where R² is H or a hydrocarbon group), and another portion of the monomer units have —YR² as —NR³R⁴R⁵. In any of the foregoing embodiments, one portion of the monomer units may be in a more predominant amount (i.e., is present in a higher number of units) than another portion of monomer units.

In particular embodiments, the polymer according to Formula (13) is an amido copolymer derivative of the polymer shown in Formula (14). In this embodiment, a portion (i.e., one or more) of the O—R² groups in the following polymer (or copolymer):

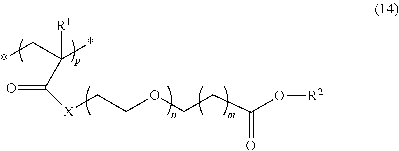

is replaced with one or more amino groups (i.e., —NR³R⁴R⁵ groups), thereby resulting in a polymer derivative wherein at least a portion of the monomer units have the following chemical structure:

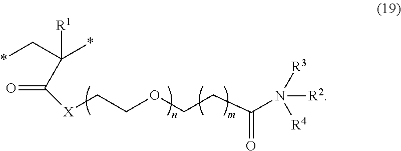

The double asterisk shown in Formula (19) indicates continuous bonding in a polymer backbone structure (i.e., *-(Formula)-* is equivalent to -(Formula)ᵣ-, where r is at least 1). The double asterisk includes the possibility that a single monomer unit according to Formula (19) is connected on each asterisk side with monomer units according to Formula (14). In the foregoing example, the amido-derivatized copolymer may contain one amido monomer unit for the entire polymer, or may contain more than one or a multiplicity of amido monomer units wherein at least one of the amido units possesses the feature of being connected on each asterisk side with monomer units according to Formula (14). The double asterisk also includes the possibility that a block of monomer units according to Formula (19) (e.g., at least 2, 3, 4, 5, 6, 7, 8, 9, or 10 monomer units according to Formula (19), or a number of p monomer units according to Formula (19)) is connected on each asterisk side with monomer units or blocks of monomer units according to Formula (14).

In another particular set of embodiments, the polymer according to Formula (13) is an amido copolymer derivative of the mercapto polymer shown in Formula (15), completely analogously as described above for the amido polymer derivative of Formula (14). In this embodiment, a portion of the S—R² groups of the polymer (or copolymer) shown in Formula (15) is replaced with one or more amino groups (i.e., —NR³R⁴R⁵ groups), thereby resulting in a polymer derivative wherein at least a portion of the monomer units have the structure shown in Formula (19).

In yet another particular set of embodiments, the polymer according to Formula (13) is a mercapto copolymer derivative of the carboxy polymer shown in Formula (14), completely analogously as described above for the amido polymer derivative of Formula (14). In this embodiment, a portion of the O—R² groups of the polymer (or copolymer) shown in Formula (14) is replaced with one or more mercapto groups (i.e., S—R² groups), thereby resulting in a polymer derivative wherein at least a portion of the monomer units have the structure shown in the following formula:

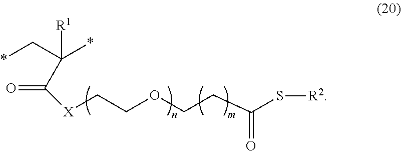

In Formula (20), X, R¹, R², n, and m are as defined above. The double asterisk has the same meaning as described above under Formula (19).

In another aspect, the invention is directed to methods for producing the polymer and copolymer compositions described above. Any of the methods known in the art for effecting addition polymerization via vinyl group coupling are applicable herein. Such methods are well known in the art. The method may employ strictly chemical means, strictly physical means (e.g., UV photolysis or ionizing radiation), or a combination thereof. Some examples of known polymerization processes include anionic polymerization, cationic polymerization, emulsion polymerization, chain growth polymerization (e.g., free radical polymerization), as well as bulk polymerization or living polymerization versions of these processes.

In particular embodiments, the polymerization method is atom transfer radical polymerization (ATRP), which is a type of living polymerization well known in the art. In ATRP, a monomer composition is subjected to radical polymerization conditions in the presence of an ATRP catalyst (typically a transition metal catalyst, such as a Cu(I) compound) and ATRP initiator (typically an alkyl halide). A particular advantage of ATRP is its ability to provide a uniform polymer chain growth (i.e., with a low polydispersity index). Other forms of ATRP, such as reverse ATRP, AGET ATRP, and ICAR ATRP, are also applicable herein.

In other embodiments, the polymerization method is Reversible Addition-Fragmentation chain Transfer (RAFT) polymerization, a controlled radical polymerization process. RAFT is particularly advantageous in the preparation of the instant polymers by virtue of its effectiveness in polymerizing a wide range of monomer compositions. Moreover, RAFT is capable of producing polymers of a specific molecular weight with very low polydispersity. RAFT is also capable of producing polymers with highly complex structures, such as comb, brush, star, and dendrimer polymers.

In addition to the monomer, the RAFT process typically employs a radical initiator, chain transfer agent, and a solvent. The initiator can be any of the initiators known in the art, but more typically an azo-containing initiator, such as azobisisobutyronitrile (i.e., AIBN) or 4,4′-azobis(4-cyanovaleric acid) (i.e., ACVA), equivalent to 4,4′-azobis(4-cyanopentanoic acid) (i.e., A-CPA), or a combination thereof. The chain transfer agent is typically a thiocarbonylthio compound (I.e., a compound containing a —C(═S)S— group). The thiocarbonylthio compound can be a dithioester, trithiocarbonate, or dithiocarbamate compound. Generally, the thiocarbonylthio agent includes a strong electronegative group (e.g., cyanide or carboxylic acid) adjacent to the thiocarbonylthio group in order for that portion of the transfer agent to function as a homolytic leaving group. Each chain transfer agent generally produces distinct polymerization results for each type of monomer, with some chain transfer agents providing significantly inferior results than others per type of monomer and the type of polymer desired. Thus, the chain transfer agent generally needs to be carefully selected to ensure effective polymerization for a particular monomer or combination of monomers. Some examples of dithioester chain transfer agents include 4-cyano-4-(thiobenzoylthio)pentanoic acid and 2-cyanoprop-2-yl-dithiobenzoate. Some examples of trithiocarbonate chain transfer agents include 2-methyl-2-[(dodecylsulfanylthiocarbonyl)sulfanyl]propanoic acid, 4-cyano-4-(dodecylsulfanylthiocarbonyl)sulfanylpentanoic acid, S-cyanomethyl-S-dodecyltrithiocarbonate, S-(2-cyanoprop-2-yl)-S-dodecyltrithiocarbonate, and S,S-dibenzyltrithiocarbonate. An example of a dithiocarbamate chain transfer agent is 2-cyanomethyl-N-methyl-N-phenyldithiocarbamate.

The RAFT process may be conducted at room temperature (i.e., about 15, 20, 25, or 30° C., or in a range therein), or at an elevated temperature (e.g., 40, 45, 50, 55, 60, 65, 70, 75, or 80° C., or in a range therein). In different embodiments, the RAFT process is practiced as a bulk, emulsion, or suspension process, conducted in either organic or aqueous solution. By the RAFT process, the cleaved portions of the chain transfer agent are retained on the terminal ends of the polymer during polymer growth, as well as in the final polymer.

The method for producing the polymer can further include steps for chemical modification of the initially produced polymer. For example, to prepare an amido copolymer derivative of the polymer shown in Formula (14) or (15), as described above, any suitable amide condensation reagent and process known in the art can be used. Some suitable amidation reagents include the carbodiimides (e.g., EDC and DCC), NHS, 1-hydroxy-7-azabenzotriazole, and hydroxybenzotriazole, as well as combinations thereof (e.g., EDC and NHS). In particular embodiments, the amide condensation reagent is 4-(4,6-dimethoxy-1,3,5-triazin-2-yl)-4-methylmorpholinium chloride (DMTMM). To prepare a mercapto copolymer derivative of the carboxy polymer shown in Formula (14), a suitable thiation agent, such as Lawesson's reagent, may be used. To modify a copolymer derivative of the polymer shown in Formula (14) when R² is H (i.e., the polymer contains carboxylic acid endcapping groups) by replacing carboxylic acid H atoms with hydrocarbon groups, an esterification process may be used, such as reaction with an alcohol under condensing conditions, or conversion of the carboxylic acid to an acyl chloride and reaction with an alcohol, or alkylation with a haloalkyl compound.

In yet another aspect, the invention is directed to a combinatorial library of the polymers described above. The combinatorial library is preferably produced by large-scale combinatorial synthetic methods, as further described in the appended Examples. The polymers in the combinatorial library can be varied (often, systematically varied) in any one or more variables selected from X, Y, R¹, R², n, m, and p. The polymers can also be varied according to the amount of derivitization (i.e., substitution) of groups in a copolymer, or stated differently, by the relative numerical or weight ratio between distinct types of monomer units. The library of polymers can be particularly useful for the purpose of high-throughput screening of the polymers to determine the effect on LCST properties of the variations made in the series of polymers. For this purpose, the combinatorial library is generally stored in or transferred to well plates (i.e., microtiter or microwell plates) widely used for combinatorial analysis and clinical diagnostics. The well plates can hold, for example, 6, 12, 24, 48, 96, 384 or 1536 sample wells, which may also correspond to the number of tested compounds. Each of the wells may hold a suitable amount of the polymer, typically in a suitable solvent. Each well typically has a volume of no more than 1 mL, 500 μL, 200 μL, 100 μL, 50 μL, 10 μL, 1 μL, 500 nL, 200 nL, or 100 nL.

By use of the combinatorial library of polymers, and their subsequent high-throughput analysis, large numbers of polymers, systematically varied in one or more variable features, can be efficiently screened for their LCST properties. Moreover, the property data can be compiled into a databank, and the data processed to elucidate structure-property relationships. At least one key advantage of such combinatorial methods is that the structure-property correlations derived therefrom are highly useful in making predictions on the properties of future hypothetical LCST polymers.

Examples have been set forth below for the purpose of illustration and to describe the best mode of the invention at the present time. However, the scope of this invention is not to be in any way limited by the examples set forth herein.

### Example 1

### Synthesis of a Carboxylic Acid-Endcapped Monomer (CTMAAm)

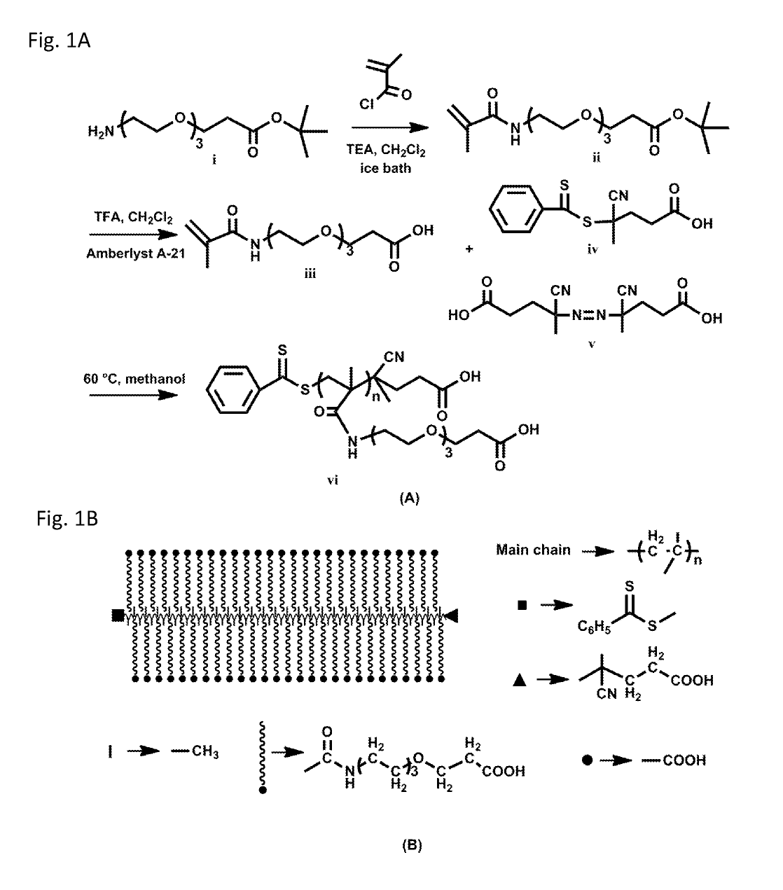

Synthesis of the CTMAAm monomer is schematically shown in FIG. 1 (A).

First, tert-butyl 12-amino-4,7,10-trioxadodecanoate (i) was reacted with methacryloyl chloride in the presence of base to form N-(tert-butyl 3,6,9-trioxado-12-decanoate) methacrylamide (ii). The details of the synthesis of compound (II) are as follows: Tert-butyl 12-amino-4,7,10-trioxadodecanoate (i) (300 mg, 0.865 mmol) and triethylamine (0.15 mL, 1.08 mmol) were dissolved in anhydrous CH₂Cl₂ (10 mL) at 0° C. under N₂ for 15 minutes. Methacryloyl chloride (0.11 mL, 1.08 mmol) in 5 mL of anhydrous CH₂Cl₂ was added dropwise in the mixture and stirred for 1 hour at 0° C. and another 2 hours at room temperature. The reaction mixture was diluted with aqueous NaHCO₃ and extracted with CH₂Cl₂. The organic phase was washed with water three times and dried over MgSO₄. The solution was filtered and concentrated at reduced pressure to produce a viscous yellow oil. Purification of the residue by flash chromatography (hexane/ethyl acetate 1:5) afforded a colorless oil of (ii), (292 mg, 80%). ¹H NMR (DMSO-d₆,): δ 1.40 (s, 9H), δ 1.84 (s, 3H), δ 2.41 (m, 2H), δ 3.26 (m, 2H), δ 3.44 (m, 2H), δ 3.50 (m, 8H), δ 3.58 (s, 2H), δ 5.32 (s, 1H), δ 5.65 (s, 1H), δ 7.94 (s, 2H). Elemental analysis: Calcd (C₁₇H₃₁NO₆): C, 59.11%; H, 9.05%; N, 4.05%. Found: C, 58.79%; H, 8.97%; N, 4.14%.

Second, CTMAAm (iii) was formed by deprotection of (ii) using trifluoroacetic acid (TFA), followed by treatment with Amberlyst A-21 to remove remaining TFA. The details of the synthesis of compound (iii) are as follows: A mixture of CH₂Cl₂ (1 mL) and trifluoroacetic acid (TFA, 1 mL) was added to 200 mg of N-(tert-butyl 3,6,9-trioxado-12-decanoate) methacrylamide (ii) in a 50 mL round-bottom flask. After stirring at room temperature for 30 minutes, the volatiles were removed in vacuo. The oil residue was dissolved in 30 mL of anhydrous CH₂Cl₂ and treated with Ig of Amberlyst A-21 resin. After stirring at room temperature for 1 hour, the solid was removed by filtration and the solvent was removed in vacuo. Purification of the residue by flash chromatography (hexane/methanol/ethyl acetate 1:0.5:20) afforded N-(12 carboxyl-3,6,9-trioxado) methacrylamide (iii, 113 mg, 57%). ¹H NMR (DMSO-d₆): δ 1.83 (s, 3H), δ 2.43 (m, 2H), δ 3.26 (m, 2H), δ 3.43 (m, 2H), δ 3.50 (m, 8H), δ 3.58 (s, 2H), δ 5.32 (s, 1H), δ 5.64 (s, 1H), δ 7.94 (s, 2H).

### Example 2

### Polymerization of CTMAAm to Form Brush-Type Polymer (pCTMAAm)

Synthesis of the pCTMAAm monomer is also schematically shown in FIG. 1 (A).

Generally, RAFT polymerization of CTMAAm was conducted at 60° C. in methanol, using 4-cyanopentanoic acid dithiobenzoate (iv) as the chain transfer agent (CTA) and 4,4′-azobis(4-cyanopentanoic acid) (v) as the radical initiator (I). The final product, poly[N-(12-carboxyl-3,6,9-trioxado)methacrylamide] (pCTMAAm) (vi), shown in picture format in FIG. 1 (B), contains a carboxyl-terminated oligomer of polyethylene glycol, which is readily soluble in a wide range of solvents.

A more detailed synthesis of the polymer (vi) via RAFT polymerization of CTMAAm is provided as follows. Prior to the experiment, all liquid reagents were purged under nitrogen for at least 10 minutes. Individual stock solutions of the radical initiator 4,4′-azobis(4-cyanopentanoic acid) (A-CPA) (iv) and 4-cyanopentanoic acid dithiobenzoate (i.e., CTA or CPA-DB) (v) were prepared with the respective solvent to ensure accurate reactant ratios. A representative example for polymerization is as follows: iii (56.38 mg, 0.244 mmol) and CPA-DB (0.247 mg, 8.9×10⁻⁴ mmol in 122 μL of methanol) were transferred into a 1 mL glass ampule equipped with a magnetic stir bar and purged under nitrogen for five minutes. Then A-CPA (0.062 mg, 2.2×10⁻⁴ mmol in 30 μL of methanol) was added into the ampule and purged under nitrogen for another two minutes. The ampule was sealed with oxygen flame and immersed in a 60° C. oil bath under continuous stirring. The reaction was stopped at 48 hours by cooling the ampule in an ice bath and then exposing the solution to air. The polymer, poly[N-(12-carboxyl-3,6,9-trioxado)methacrylamide](pCTMAAm) (vi), was obtained by precipitation in a generous amount of stirring diethyl ether, filtered, and dried under vacuum overnight. The polymer was further purified by dialysis using Spectra/Pro regenerated cellulose dialysis tubing (3.5 kDa MWCO) against deionized-water for three days and lyophilized for 2 days. Mₙ and PDI calculated by GPC for this sample were 46,100 Da and 1.07, respectively, and the percent conversion, estimated by gravimetric analysis, was 87%. ¹H NMR (400 MHz, D₂O, ppm): δ 0.92 (br s, 3H), δ 1.65 (br s, 2H), δ 2.67 (m, 2H), δ 3.32 (m, 2H), δ 3.58 (m, 2H), δ 3.68 (m, 8H), δ 3.80 (s, 2H), δ 7.71 (s, 2H).

Several pCTMAAm polymers were prepared using different initial concentrations of monomer ([M]₀) and different ratios of [M]₀/[CTA]₀ whereas the [CTA]₀/[I]₀ ratio was held constant (1:0.25), as shown in Table 1 below. Owing to the efficient reversible addition-fragmentation chain transfer ability of iv, and the coordination of monomer-initiator pair in this system, well-defined pCTMAAm polymers were obtained with different molecular weights consistent with theoretical molecular weights and with very narrow polydispersity (PDI=1.05-1.09). By adjusting the [M]₀/[CTA]₀ ratio, several number average molecular weights (Mₙ) of pCTMAAms were obtained ranging from 10100 up to 84300 in accordance with the theoretical Mₙ at the same [M]₀ of 1.5 mol L⁻¹. These results are consistent with the controlled behavior of RAFT polymerization. The polymerization yields were high (˜80-90%) with the exception of the low yield (53%) at extremely low [CTA]₀. [M]₀ had no obvious effect on PDI comparing three different [M]₀ (1.0, 1.5 and 2.0 mol L¹), and the highest conversion and Mₙ were obtained at [M]₀ of 2.0 mol L¹.

TABLE 1
RAFT polymerization of pCTMAAm under
different reaction conditions.

| | | | | | | Mₙ^{c} |
|---|---|---|---|---|---|---|
| | [M]₀/ | [M]₀ | Mₙ^{a} | | Yield^{b} | Theory |
| entry | [CTA]₀ | mol L⁻¹ | g mol⁻¹ | PDI^{a} | % | g mol⁻¹ |
| 1 | 45 | 1.5 | 10100 | 1.09 | 89 | 10700 |
| 2 | 70 | 1.5 | 14400 | 1.08 | 82 | 16400 |
| 3 | 120 | 1.5 | 29600 | 1.06 | 81 | 28300 |
| 4 | 170 | 1.5 | 41300 | 1.05 | 80 | 39500 |
| 5 | 200 | 1.5 | 47400 | 1.07 | 82 | 47700 |
| 6 | 300 | 1.5 | 52700 | 1.06 | 79 | 68700 |
| 7 | 600 | 1.5 | 84300 | 1.06 | 53 | 91900 |
| 8 | 200 | 1.0 | 42300 | 1.07 | 78 | 45100 |
| 9 | 200 | 2.0 | 50800 | 1.07 | 80 | 46500 |

^{a}By GPC.
^{b}By gravimetric analysis.
^{c}Mₙ (theory) calculated as previously described (Brouwer, H. D., et al., J. Polym. Sci. Polym. Chem. Ed., 38, 3596-3603 (2000); Pelet, J. M., et al., Macromolecules, 42, 1494-1499 (2009).

The solubility of pCTMAAm polymer in different solvents is provided in Table 2 below. PP-46.12

TABLE 2
Solubility of pCTMAAm polymer in different solvents

| | | | | | Tetra- | | |
|---|---|---|---|---|---|---|---|
| Wa- | Meth- | Eth- | Dimethyl | Dimethyl | hydro- | | Ace- |
| ter | anol | anol | sulfoxide | formamide | furan | CHCl₃ | tone |
| S | S | S | S | S | SS | I | I |

S, soluble (up to a concentration of at least 20 g/L);
SS, slightly soluble;
I, insoluble.

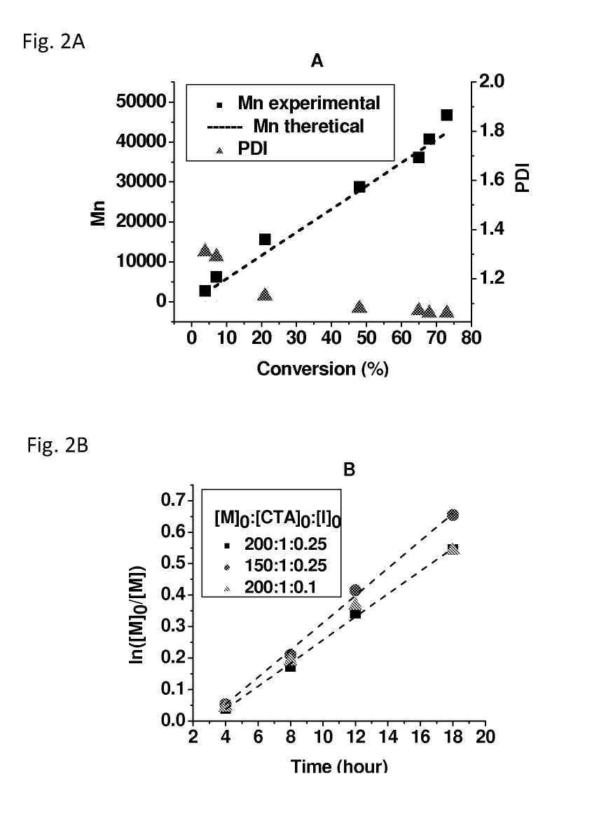

To identify the controlled/living characteristic of the RAFT polymerization of CTMAAm, the relationship of monomer conversion versus Mₙ and PDI of the polymer were studied. A series of RAFT polymerizations ([M]₀/[CTA]₀/[I]₀=200:1:0.25) were conducted for 4, 8, 12, 18, 24, 36, and 48 hours in methanol with [M]₀=1.5 mol L⁻¹. As shown in FIG. 2 (A), Mₙ (GPC) agreed with the Mₙ (theory) over the course of the polymerization with substantial linearity. At low conversion, the PDI is relatively high, but decreased from 1.31 to 1.06 as Mₙ increased owing to the gradual attainment of chain transfer equilibrium. Varying the reactant ratio of [M]₀/[CTA]₀/[I]₀ with comparison of 200:1:0.25, 150:1:0.25 and 200:1:0.1 (FIG. 2B), the three polymerizations developed linearly with time. Pseudo-first-order kinetics were observed for the RAFT polymerizations with a slight induction time (about 2 hours) for all the polymerization conditions. At the same [CTA]₀/[I]₀ ratio, decreasing [M]₀/[CTA]₀ caused an increase in polymerization rate due to a higher relative concentration of CTA active species. However, decreasing the radical initiator concentration 2.5-fold did not have a significant impact on polymerization rate.

Solubility assessment showed that pCTMAAm is readily soluble in a range of solvents (defined as >20 g/L) including water, methanol, ethanol, DMSO and DMF. Additionally, dynamic light scattering showed that the polymer was in an extended conformation in each solvent, suggesting that the side chain carboxyl termini would be synthetically accessible. Using three model ligands, agmatine (cationic), galactosamine (polyol) and hexylamine (hydrophobic) and DMTMM as a condensation agent, the functionalization characteristics of pCTMAAm was determined. Each ligand type was readily accepted by the carboxyl groups of pCTMAAm. With a target substitution of 100%, the substitution yield for each ligand exceeded 80%. Specifically, agmatine yielded 83% (reaction in water), galactosamine yielded 80% (reaction in water), and hexylamine yielded 94% (reaction in methanol) and 82% (reaction in DMSO). As a comparison, the substitution of agmantine, galactosamine and hexylamine to poly(methacrylic acid) with the same molecular weight resulted in insoluble products under all reaction conditions indicating that the oligomer ethylene oxide side chains of pCTMAAm also act as solubilizing facilitators in a range of solvents.

Thus, a new precursor for the synthesis of functional biomaterial libraries has been described. The monomer is easily polymerized via RAFT polymerization with narrow PDI and controllable molecular weight. pCTMAAm, in particular, has side chains terminated with carboxyl groups, which are readily functionalized in both protic and aprotic solvents to allow for the facile substitution of a range of functional groups and increase the potential diversity of a polymer library.

### Example 3

### Combinatorial Polymeric Libraries by Derivitization of pCTMAAm as Template

Efforts to develop polymers having a specified lower critical solution temperature (LCST) have largely relied on empirical means. As empirical means are substantially based on trial and error, and a diverse set of variables are at work in determining polymer properties, such means are significantly inefficient in attempting to find LCST polymers having specific properties. Thus, the instant combinatorial work has been designed in an effort to find polymers with specific LCST characteristics in a more directed manner. Specifically, the instant research seeks to systematically vary one or more structural variables of LCST polymers described herein to produce a library of such polymers, and test the library of polymers by high-throughput screening methods. Moreover, the data garnered by such studies can be entered into a database, and the data analyzed to elucidate structure-property correspondences, which can then also be useful as a predictive tool in predicting the LCST properties of untested polymers.

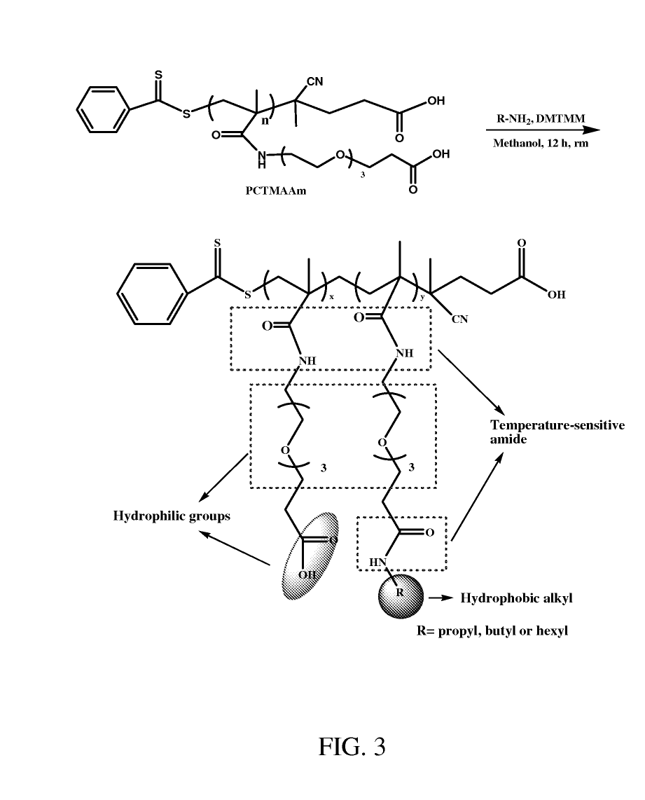

In an exemplary study detailed herein, a library of 45 LCST polymers were studied. The 45 LCST polymers were made to vary in the following variables: the molecular weight of the polymers, the size of the endcapping hydrophobic substituent (i.e., at R²), and the degree of substitution of the hydrophobic groups in the polymer (i.e., the relative number of initial R² groups substituted by hydrocarbon groups). The polymers in the polymer library were prepared in parallel under equivalent reaction conditions in order to prevent the occurrence of unintended structural differences caused by differences in preparative conditions. As further shown by the preparative scheme in FIG. 3, pCTMAAm (having carboxylic acid endcapping groups, i.e., where —OR² is —OH) was employed as the polymer precursor on which was conjugated different alkyl groups via a facile condensation reaction with 4-(4,6-Dimethoxy-1,3,5-triazin-2-yl)-4-methylmorpholinium chloride (DMTMM) as catalyst. As shown in FIG. 3, the conjugated polymer is a copolymer containing x monomer units derived from the initial pCTMAAm polymer, as well as y monomer units wherein OH groups of the endcapping carboxylic acid groups of the pCTMAAm polymer have been replaced with amino groups (—NHR, where R has the same meaning as R²). Thus, the substituted copolymer contains both carboxylic acid endcapping groups (which are substantially hydrophilic) and N-substituted carboxamide endcapping groups (—C(O)NHR), where R is an alkyl group, varied in the number of carbon atoms (specifically, n-propyl, n-butyl, and n-hexyl), which are substantially hydrophobic. As summarized in Table 3 below, three different molecular weights were also varied, as well as a number of substitution levels.

TABLE 3
Structural features varied in LCST polymers

| Structural Variable | Variation |
|---|---|
| Molecular weights of pCTMAAms | 15,000 (PDI = 1.14); 38,000 |
| | (PDI = 1.07); 56,000 (PDI = 1.08) |
| Substituted group R | propyl, butyl, hexyl |
| Total R substitution levels (e.g., | 4 for propyl, 5 for butyl, |
| mol % range) | 6 for hexyl |
| Total number of samples synthesized | 45 |

Prior to conjugation, three pCTMAAm polymers with different molecular weight (as summarized in Table 3) were prepared by RAFT polymerization to provide very low PDI (<1.15) according to the method described above. The conjugation is facile as follows: pCTMAAm and 4-(4,6-dimethoxy[1,3,5]triazin-2-yl)-4-methylmorpholiniumchloride (DMTMM) were dissolved in methanol prior to the additional of alkyl amines, with a specified molar ratio of [COOH in pCTMAAm]:[DMTMM]:[NH₂ in alkyl amines (RNH₂)], and the initial concentration of pCTMAAm at 20 mg/mL. DMTMM was employed as the condenser to couple carboxyl group with amine group and form amines due to its excellent solubility in water and alcohols, high efficiency, and lack of byproducts. After 18 hours of stirring at room temperature, the modified LCST polymers were obtained with the removal of methanol in vacuo. The ratios of [DMTMM]:[NH₂ in alkyl amines] were kept at 1:1 to provide the same activity of amino groups. The LCST polymers with different substitution from 23%-90% were obtained by adjusting the reaction ratio of [COOH in pCTMAAm]:[NH₂ in alkyl amines]. In order to further purify the conjugated products, the products were dissolved in water and dialyzed against de-ionized water for three days with three changes of de-ionized water each day, then lyophilized for two days. By this relatively facile methodology, a full polymer library was typically prepared within one week.

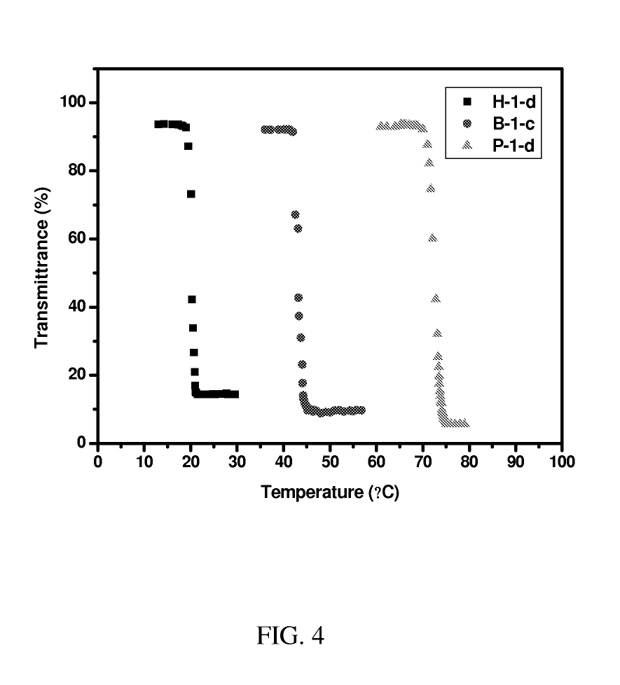

Once the polymer library was prepared, a high-throughput screening of LCST test was conducted as follows. Each polymer in the library was dissolved in de-ionized (DI) water to 3 mg/mL concentration and transferred to a 96-well plate having 200 μL well volumes, then tested by use of a microplate spectrophotometer reading at 500 nm with a continuous temperature conversion from 2° C. to 90° C. The LCST results are shown in the transmittance vs. temperature graph shown in FIG. 4. As shown in FIG. 4, a wide range of LCST from 4° C. to 85° C. was observed in the polymer library. The LCST was defined as the midpoint of the temperature-transmission curve. The sharp transition exhibited for each sample demonstrated a remarkable temperature sensitivity in these polymers. The LCST tests were conducted in triplicate in order to verify the repeatability of the data. The LCST tests conducted herein were typically completed in about one or two days.

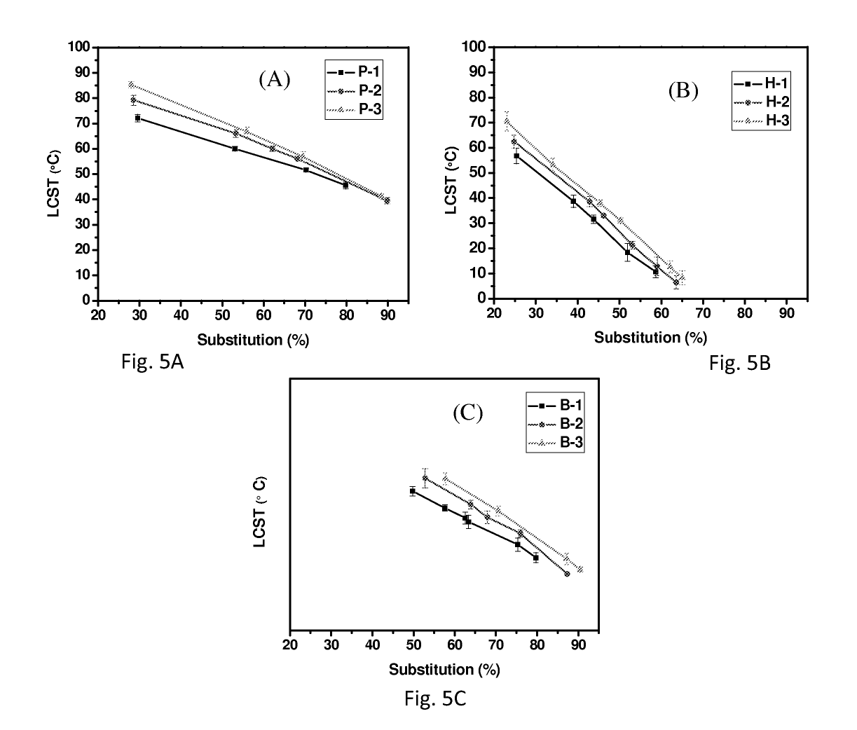

The relationship between the LCST and three structure parameters (the molecular weight of template polymer, the length of conjugated alky groups, and the degree of conjugation substitution) are presented in FIGS. 5A-5C. As shown by FIGS. 5A-5C, whether the conjugation group is propyl, butyl, or hexyl, the LCST of polymers with the same molecular weight exhibited an almost linear decrease with increase in the degree of conjugation. This is believed to be due to the increasing hydrophobicity of the polymer system with increase in the degree of conjugation substitution. The increase in hydrophobicity allows the polymer to reach the critical point of hydrophilic-hydrophobic interaction with less energy to overcome the hydrogen bonds between amide groups and water molecules. As also shown by FIGS. 5A-5C, at the same substitution degree, the higher the molecular weight of the polymer, the lower the LCST it has, which is found to be the case whether the endcapping hydrocarbon group is propyl, butyl or hexyl. The latter effect is believed to be due to the tendency of higher molecular weight polymers to be less able to freely extend itself, and moreover, the tendency to aggregate with itself to form globules (typically exhibited as solid precipitates in solution at lower temperature). As further shown by FIGS. 5A-5C, with molecular weight and substitution held constant, the polymers showed decreasing LCSTs with growth of conjugated alky chain length from propyl (3 carbons), butyl (4 carbons), to hexyl (6 carbons). The latter effect is believed to be due the increasing ability of longer chain alky groups to make the polymer reach the phase transition at lower temperature, thereby corresponding to a general trend of decreasing LCST with increasing alkyl chain length. Furthermore, regardless of the molecular weight of template polymers, the slopes of the three substitution dependent LCST curves increased with increase in the carbon number in the conjugated alkyl groups from propyl, butyl, to hexyl, which indicates that the longer alky chain exerts more influence on the LCSTs of polymers with the same conjugation substitution.

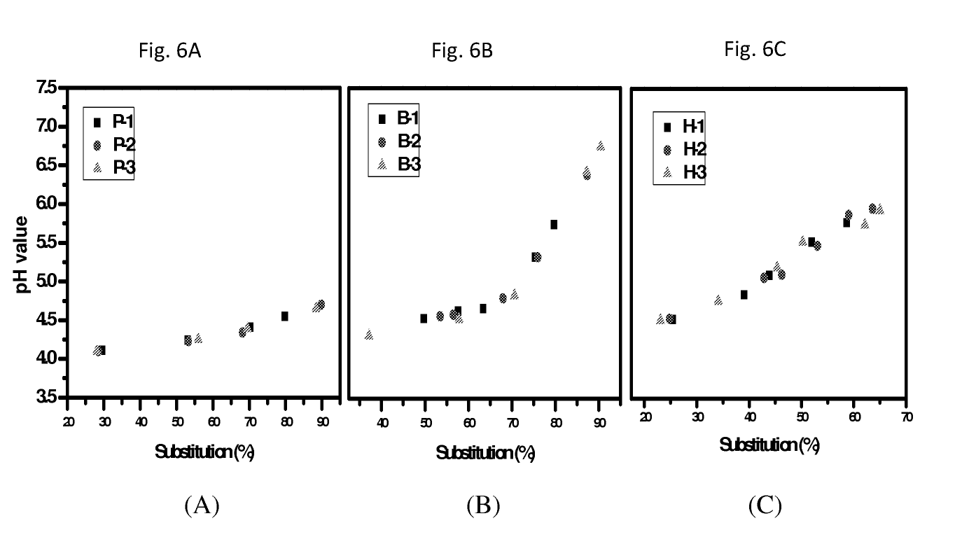

As shown by FIGS. 6A-C, regardless of propyl, butyl, or hexyl substituent in the conjugated group, the pH value of the polymer solution increased with increase in the degree of conjugation substitution. The latter effect is believed to be due to the decrease in stabilizing carboxyl-carboxyl hydrogen bond interaction in polymers increasingly conjugated with hydrophobic NHR groups. As also shown by FIGS. 6A-C, at the same degree of conjugation substitution, the pH value increases with increase in the length of conjugated alkyl group from propyl, butyl, to hexyl. For example, at a substitution of 60%, the pH value for propyl, butyl, and hexyl series are, respectively, around 4.25, 4.5 and 5.5. The latter effect is believed to be due to the decrease in stabilizing carboxyl-carboxyl hydrogen bond interaction in polymers with endcapping R groups of increasing length (also due to increase in hydrophobic-hydrophobic interactions in R groups of increasing length). As shown, these LCST polymers are substantially pH-sensitive.

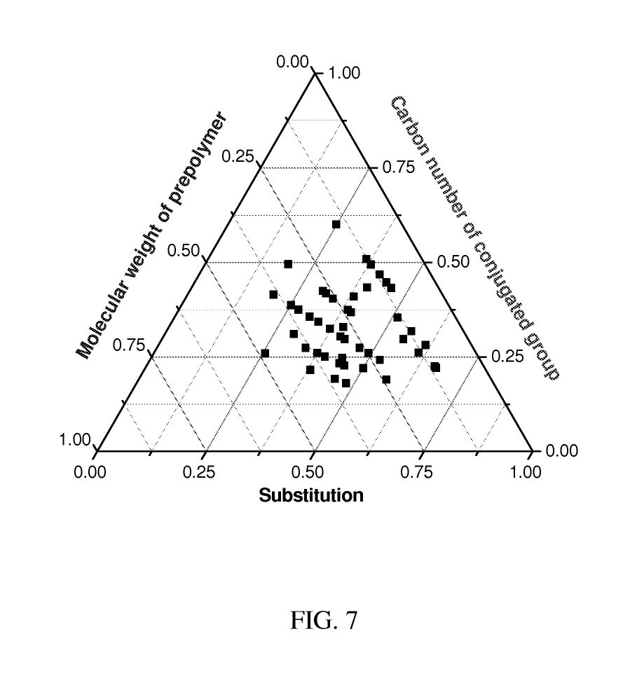

Based on the data from this 45-member polymer library, a three-phase diagram of LCST polymers with three parameter spaces, including the molecular weight of template polymer, the substitution degree, and the carbon number of conjugation group, was produced. The three-phase diagram is shown in FIG. 7. The data was normalized using OriginPro 8.0 to fit to the triangular phase diagram. In order to adjust polymers distributed in the center of the graph, the data used as the substitution degree are the percent of the original data; the data used as the carbon number of conjugation group are the tenfold of the actual number; and the data used in the molecular weight of the polymer are one-thousandth of the actual data.

The three factors, the molecular weight of polymers, the carbon number of conjugation group, and the substitution degree, coordinately determines the position of a polymer in the three-phase diagram shown in FIG. 7. The diagram in FIG. 7 makes it possible to predict the LCST of a hypothetical polymer by inputting structural parameters of the hypothetical polymer into the program and observing its position with respect to known LCST values. For example, if the molecular weight of the template polymer is 60×10³, the carbon number of conjugation group is 5, and the substitution is 40%, then the normalized data of the three axes should be 0.4, 0.33 and 0.27. The position marked with an arrow in FIG. 7 shows where the hypothetical LCST polymer would fall in the diagram. Thus, the three-phase diagram can be used as a highly useful predictive tool in finding new LCST polymers with special LCST values along with other unique properties.

The raw data used in generating the three-phase diagram in FIG. 7 is provided in Table 4 below.

TABLE 4
Raw data used for generating the three-phase diagram in FIG. 7.

| | Substi- | | | Substi- | | | Substi- | |
|---|---|---|---|---|---|---|---|---|
| Sam- | tution | | Sam- | tution | | Sam- | tution | |
| ple | (%) | pH | ple | (%) | pH | ple | (%) | pH |
| P-1-a | 79.84 | 4.55 | P-2-a | 89.85 | 4.7 | P-3-a | 88.37 | 4.65 |
| P-1-b | 70.24 | 4.41 | P-2-b | 68.15 | 4.34 | P-3-b | 69.51 | 4.39 |
| P-1-c | 53.05 | 4.24 | P-2-c | 53.27 | 4.23 | P-3-c | 55.95 | 4.25 |
| P-1-d | 29.58 | 4.11 | P-2-d | 28.57 | 4.10 | P-3-d | 28.06 | 4.10 |
| B-1-a | 79.72 | 5.74 | B-2-a | 87.29 | 6.38 | B-3-a | 90.42 | 6.74 |
| B-1-b | 75.31 | 5.32 | B-2-b | 75.96 | 5.32 | B-3-b | 87.20 | 6.42 |
| B-1-c | 63.39 | 4.66 | B-2-c | 67.95 | 4.79 | B-3-c | 70.59 | 4.83 |
| B-1-d | 57.63 | 4.63 | B-2-d | 56.52 | 4.58 | B-3-d | 57.81 | 4.52 |
| B-1-e | 49.75 | 4.53 | B-2-e | 53.49 | 4.56 | B-3-e | 37.11 | 4.31 |
| H-1-a | 68.65 | 6.06 | H-2-a | 65.03 | 5.95 | H-3-a | 63.60 | 5.93 |
| H-1-b | 58.68 | 5.77 | H-2-b | 62.12 | 5.87 | H-3-b | 59.02 | 5.74 |
| H-1-c | 51.92 | 5.52 | H-2-c | 50.25 | 5.47 | H-3-c | 53.05 | 5.62 |
| H-1-d | 43.82 | 5.09 | H-2-d | 45.36 | 5.10 | H-3-d | 46.24 | 5.19 |
| H-1-e | 39.02 | 4.84 | H-2-e | 43.20 | 5.06 | H-3-e | 42.86 | 4.76 |
| H-1-f | 28.57 | 4.52 | H-2-f | 23.07 | 4.53 | H-3-f | 24.81 | 4.51 |

While there have been shown and described what are at present considered the preferred embodiments of the invention, those skilled in the art may make various changes and modifications which remain within the scope of the invention defined by the appended claims.

1. A monomer composition represented by the following chemical structure: 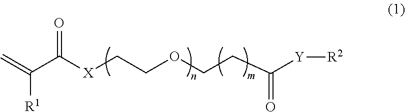
wherein R¹ and R² are independently selected from a hydrogen atom or a hydrocarbon group containing at least one carbon atom; X represents an —O— or —NR⁵— group, and Y represents an —O—, —S—, or —NR³R⁴— group, wherein R³, R⁴, and R⁵ independently represent a hydrogen atom or a hydrocarbon group containing at least one carbon atom, except that one of R³ and R⁴ can instead represent an unshared pair of electrons; the subscript n represents an integer of at least 4; and the subscript m represents 0 or an integer of at least 1; wherein said hydrocarbon group is independently and optionally substituted by at least one heteroatom or heteroatom group; wherein R² can alternatively be comprised of a biological species; and wherein, when Y is an —O— or —S— group, the bond shown between R² and Y is either a covalent or ionic bond, provided that if said bond is ionic, R² is an organic or inorganic cationic group that counterbalances a negative charge on Y; and wherein the group —Y—R² can, itself, be a cationic group that is necessarily associated with an anionic counterion; or a salt of said monomer composition.

2.-12. (canceled)

13. The monomer composition of claim 1, wherein R¹ is a hydrogen atom or methyl group.

14. The monomer composition of claim 1, wherein subscript n represents an integer of at least 5.

15. The monomer composition of claim 1, wherein subscript m represents an integer of at least 1.

16. (canceled)

17. A monomer composition represented by the following chemical structure: 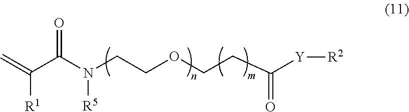
wherein R¹ and R² are independently selected from a hydrogen atom or a hydrocarbon group containing at least one carbon atom; Y represents an —O—, —S—, or —NR³R⁴— group, wherein R³, R⁴, and R⁵ independently represent a hydrogen atom or a hydrocarbon group containing at least one carbon atom, except that one of R³ and R⁴ can instead represent an unshared pair of electrons; the subscript n represents an integer of at least 1; and the subscript m represents 0 or an integer of at least 1; wherein said hydrocarbon group is independently and optionally substituted by at least one heteroatom or heteroatom group; wherein R² can alternatively be comprised of a biological species; and wherein, when Y is an —O— or —S— group, the bond shown between R² and Y is either a covalent or ionic bond, provided that if said bond is ionic, R² is an organic or inorganic cationic group that counterbalances a negative charge on Y; and wherein the group —Y—R² can, itself, be a cationic group that is necessarily associated with a anionic counterion; or a salt of said monomer composition; with the proviso that the following compounds are excluded: a monomer composition according to Formula (3) in which R¹ is methyl, n is 3, m is 0, Y is O, and R² is H; a monomer composition according to Formula (3) in which R¹ is methyl, n is 3, m is 0, Y is O, and R² is ethyl; and a monomer composition according to Formula (3) wherein R¹ is H, n is 3, m is 0, Y is O, and R² is t-butyl.

18. The monomer composition of claim 17, wherein subscript n represents an integer of at least 4.

19. The monomer composition of claim 17, wherein subscript m represents an integer of at least 1.

20. (canceled)

21. The monomer composition of claim 17, wherein Y represents an —O— group.

22. The monomer composition of claim 17, wherein Y represents an —S— group.

23. The monomer composition of claim 17, wherein Y represents an —NR³R⁴— group.

24. A monomer composition represented by the following chemical structure: 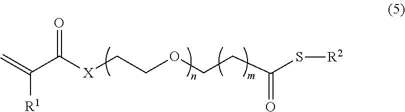
wherein R¹ and R² are independently selected from a hydrogen atom or a hydrocarbon group containing at least one carbon atom; X represents an —O— or —NR⁵— group, wherein R⁵ represents a hydrogen atom or a hydrocarbon group containing at least one carbon atom; the subscript n represents an integer of at least 1; and the subscript m represents 0 or an integer of at least 1; wherein said hydrocarbon group is independently and optionally substituted by at least one heteroatom or heteroatom group; wherein R² can alternatively be comprised of a biological species; and wherein the bond shown between R² and S can be a covalent or ionic bond, provided that if said bond is ionic, R² is an organic or inorganic cationic group that counterbalances a negative charge on S.

25. The monomer composition of claim 24, wherein X is —O—.

26. The monomer composition of claim 24, wherein X is —NR⁵—.

27. The monomer composition of claim 24, wherein n is at least 2.

28. (canceled)

29. The monomer composition of claim 24, wherein m is at least 1.

30. A monomer composition represented by the following chemical structure: 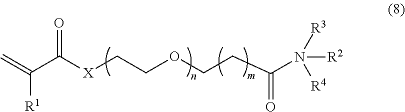
wherein R¹, R², R³, and R⁴ are independently selected from a hydrogen atom or a hydrocarbon group containing at least one carbon atom, except that one of R², R³, and R⁴ can instead represent an unshared pair of electrons; X represents an —O— or —NR⁵— group, wherein R⁵ represents a hydrogen atom or a hydrocarbon group containing at least one carbon atom; the subscript n represents an integer of at least 1; and the subscript m represents 0 or an integer of at least 1; wherein said hydrocarbon groups are independently and optionally substituted by at least one heteroatom or heteroatom group; wherein R² can alternatively be comprised of a biological species; and wherein, if none of R², R³, and R⁴ represents an unshared pair of electrons, then the nitrogen atom bonded to the groups R², R³, and R⁴ is positively charged and necessarily is associated with a counteranion.

31. The monomer composition of claim 30, wherein X is —O—.

32. The monomer composition of claim 30, wherein X is —NR⁵—.

33. The monomer composition of claim 30, wherein n is at least 2.

34. (canceled)

35. The monomer composition of claim 30, wherein m is at least 1.

36. The monomer composition of claim 30, wherein at least one of R², R³, and R⁴ is a hydrocarbon group substituted by at least one hydrophilic group.

37. The monomer composition of claim 36, wherein said at least one hydrophilic group is selected from the group consisting of amino, imino, amido, hydroxyl, ether, polyether, carboxyl, ester, carbamato, ureido, aldehydro, keto, sulfate, sulfonate, sulfone, sulfoxide, sulfite, phosphate, phosphonate, phosphinate, phosphite, nitro, nitroso, and charged groups.

38. The monomer composition of claim 30, wherein at least one of R², R³, and R⁴ is a hydrocarbon group that contains solely carbon and hydrogen atoms, and optionally, one or more halogen atoms.

39. The monomer composition of claim 38, wherein said hydrocarbon group contains at least two carbon atoms.

40. The monomer composition of claim 38, wherein said hydrocarbon group contains at least three carbon atoms.

41. The monomer composition of claim 30, wherein at least one of R², R³, and R⁴ is an amphiphilic group comprising a hydrophobic moiety and a hydrophilic moiety.

42. The monomer composition of claim 41, wherein said amphiphilic group is comprised of a hydrophobic linking moiety endcapped by at least one hydrophilic group.

43. A polymer composition comprising the following chemical structure: 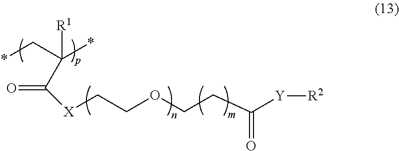
wherein R¹ and R² are independently selected from a hydrogen atom or a hydrocarbon group containing at least one carbon atom; X represents an —O— or —NR⁵— group, and Y represents an —O—, —S—, or —NR³R⁴— group, wherein R³, R⁴, and R⁵ independently represent a hydrogen atom or a hydrocarbon group containing at least one carbon atom, except that one of R³ and R⁴ can instead represent an unshared pair of electrons; the subscript n represents an integer of at least 1; the subscript m represents 0 or an integer of at least 1; and the subscript p represents an integer of at least 2; wherein said hydrocarbon group is independently and optionally substituted by at least one heteroatom or heteroatom group; wherein R² can alternatively be comprised of a biological species; and wherein, when Y is an —O— or —S— group, the bond shown between R² and Y is either a covalent or ionic bond, provided that if said bond is ionic, R² is an organic or inorganic cationic group that counterbalances a negative charge on Y; and wherein the group —Y—R² can, itself, be a cationic group that is necessarily associated with a anionic counterion; or a salt of said polymer composition, or a copolymer of said polymer composition.

44. The polymer of claim 43, wherein p is at least 10.

45.-49. (canceled)

50. The polymer of claim 43, wherein the polymer possesses a weight average molecular weight of at least 1,000.

51. The polymer of claim 43, wherein the polymer possesses a polydispersity value greater than 2.

52. The polymer of claim 43, wherein the polymer possesses a polydispersity value less than 1.5.

53.-54. (canceled)

55. The polymer composition of claim 43, wherein Y represents an —O— group.

56.-63. (canceled)

64. An amido copolymer derivative of the polymer of claim 55, wherein a portion of the O—R² groups have been replaced with amino groups, thereby resulting in a polymer derivative wherein at least a portion of the monomer units have the following chemical structure: 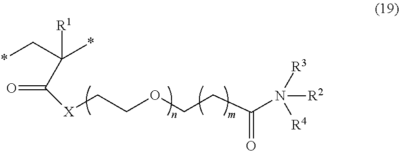
wherein R¹, R², R³, and R⁴ are independently selected from a hydrogen atom or a hydrocarbon group containing at least one carbon atom, except that one of R², R³, and R⁴ can instead represent an unshared pair of electrons; X represents an —O— or —NR⁵— group, wherein R⁵ represents a hydrogen atom or a hydrocarbon group containing at least one carbon atom; the subscript n represents an integer of at least 1; and the subscript m represents 0 or an integer of at least 1; wherein said hydrocarbon groups are independently and optionally substituted by at least one heteroatom or heteroatom group; wherein R² can alternatively be comprised of a biological species; and wherein, if none of R², R³, and R⁴ represents an unshared pair of electrons, then the nitrogen atom bonded to the groups R², R³, and R⁴ is positively charged and necessarily is associated with a counteranion; or a salt of said polymer composition.

65. (canceled)

66. A mercapto copolymer derivative of the polymer of claim 55, wherein a portion of the O—R² groups have been replaced with mercapto groups, thereby resulting in a polymer derivative wherein at least a portion of the monomer units have the following chemical structure: 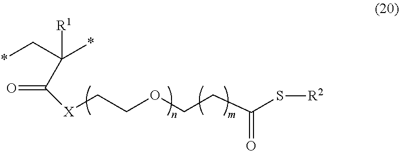
wherein R¹ and R² are independently selected from a hydrogen atom or a hydrocarbon group containing at least one carbon atom; X represents an —O— or —NR⁵— group, wherein R⁵ represents a hydrogen atom or a hydrocarbon group containing at least one carbon atom; the subscript n represents an integer of at least 1; and the subscript m represents 0 or an integer of at least 1; wherein said hydrocarbon group is independently and optionally substituted by at least one heteroatom or heteroatom group; wherein R² can alternatively be comprised of a biological species; and wherein the bond shown between R² and S can be a covalent or ionic bond, provided that if said bond is ionic, R² is an organic or inorganic cationic group that counterbalances a negative charge on S; or a salt of said polymer composition.

67. A copolymer derivative of the polymer of claim 55 when R² is H, wherein at least a portion of the R² groups have been replaced with hydrocarbon groups.

68. A method for producing a polymer according to claim 43, the method comprising polymerization of a monomer composition having the following structure: 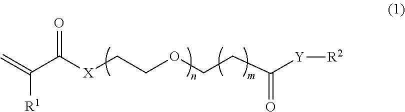
wherein R¹ and R² are independently selected from a hydrogen atom or a hydrocarbon group containing at least one carbon atom; X represents an —O— or —NR⁵— group, and Y represents an —O—, —S—, or —NR³R⁴— group, wherein R³, R⁴, and R⁵ independently represent a hydrogen atom or a hydrocarbon group containing at least one carbon atom, except that one of R³ and R⁴ can instead represent an unshared pair of electrons; the subscript n represents an integer of at least 1; and the subscript m represents 0 or an integer of at least 1; wherein said hydrocarbon group is independently and optionally substituted by at least one heteroatom or heteroatom group; and wherein, when Y is an —O— or —S— group, the bond shown between R² and Y is either a covalent or ionic bond, provided that if said bond is ionic, R² is an organic or inorganic cationic group that counterbalances a negative charge on Y; and wherein the group —Y—R² can, itself, be a cationic group that is necessarily associated with a anionic counterion; or a salt of said monomer composition.

69. The method of claim 68, wherein said polymerization is free radical polymerization.

70. The method of claim 69, wherein said free radical polymerization is RAFT polymerization, wherein, in said RAFT polymerization, a monomer composition according to claim 1 is subjected to radical polymerization conditions in the presence of at least one thiocarbonylthio chain transfer agent and a radical initiator.

71. The method of claim 69, wherein said free radical polymerization is ATRP polymerization, wherein, in said ATRP polymerization, a monomer composition according to claim 1 is subjected to radical polymerization conditions in the presence of an ATRP catalyst and ATRP initiator.

72. A method for producing a copolymer, the method comprising:
(i) polymerization of a monomer composition according to the process delineated in claim 68 where Y is —O— and R² is H to produce a precursor polymer having carboxylic acid end groups; and
(ii) replacing a portion of OH groups in said carboxylic acid end groups with functional groups selected from amino groups, thiol groups, and alkoxide groups.

73. A method for producing an amido copolymer derivative according to claim 64, the method comprising:
(i) polymerization of a monomer composition according to the process delineated in claim 68 where Y is —O— to produce a precursor polymer; and
(ii) functionalizing said precursor polymer with amido groups to produce an amido polymer derivative wherein at least a portion of the O—R² groups of said precursor polymer have been replaced with amino groups, thereby resulting in an amido polymer derivative wherein at least a portion of the monomer units have the chemical structure shown in claim 64.

74. The method of claim 73, wherein the functionalization with amido groups is achieved by employing an amide condensation agent.

75. The method of claim 74, wherein the amide condensation agent is 4-(4,6-dimethoxy-1,3,5-triazine-2-yl)-4-methylmorpholinium chloride (DMTMM).

76. A combinatorial library of LCST polymers of claim 43, wherein said LCST polymers vary in one or more variables selected from X, Y, R¹, R², n, m, and p.

77. A method for high-throughput screening of a combinatorial library of LCST polymers of claim 43 to elucidate their LCST properties, the method comprising subjecting said combinatorial library of LCST polymers to high-throughput spectrophotometric analysis useful in determining said LCST properties.
